(19) United States
(12) Patent Application Publication
(10) Pub. No.: US 2025/0295668 A1
(43) Pub. Date: Sep. 25, 2025

(54) METHODS FOR INDUCING COLANIC ACID BIOSYNTHESIS

(71) Applicant: Howard Hughes Medical Institute, Ashburn, VA (US)

(72) Inventors: Meng Wang, Ashburn, VA (US); Guo Hu, Ashburn, VA (US)

(73) Assignee: Howard Hughes Medical Institute, Ashburn, VA (US)

(21) Appl. No.: 19/084,564

(22) Filed: Mar. 19, 2025

Related U.S. Application Data
(60) Provisional application No. 63/567,616, filed on Mar. 20, 2024.

(51) Int. Cl. A61K 31/546; A61K 31/05; A61K 31/155; A61K 31/216; A61K 31/352; A61K 31/4045; A61K 31/4706; A61K 31/496; A61K 31/519; A61K 31/69; A61K 35/00; A61K 35/741; A61P 3/00

(52) U.S. Cl. CPC A61K 31/546; A61K 31/05; A61K 31/155; A61K 31/216; A61K 31/352; A61K 31/4045; A61K 31/4706; A61K 31/496; A61K 31/519; A61K 31/69; A61K 35/741; A61P 3/00; A61K 2035/115

(57) ABSTRACT

Disclosed herein are methods for inducing colanic acid production in E. coli by contacting an E. coli bacterium with cephaloridine at a concentration below the minimum inhibitory concentration for growth, wherein ZraS mediates the induction of colanic acid production in the E. coli bacterium. Also disclosed herein are methods for treating age-related metabolic dysfunction, and in particular age-related metabolic dysfunction characterized by elevated insulin and elevated low density lipoprotein (LDL) cholesterol levels relative to high density lipoprotein (HDL) levels.

### CROSS-REFERENCE TO RELATED APPLICATIONS

This application claims the benefit of and priority to U.S. Provisional Application No. 63/567,616, filed Mar. 20, 2024, the contents of which are incorporated herein by reference in their entireties.

### TECHNICAL FIELD

The present technology relates to methods and for inducing colanic acid biosynthesis in a bacterium. In particular, the present technology relates to methods of inducing ZraS-mediated colanic acid production in E. coli by contacting E. coli with concentrations of cephaloridine below the minimum inhibitory concentration. The present technology also relates to methods for treating age-related metabolic dysfunction, and in particular age-related metabolic dysfunction characterized by elevated insulin and elevated low density lipoprotein (LDL) cholesterol levels relative to high density lipoprotein (HDL) levels.

### BACKGROUND

The following description is provided to assist the understanding of the reader. None of the information provided or references cited is admitted to be prior art to the methods disclosed herein.

Colanic acid is a bacterial metabolite, which has been reported to have longevity-enhancing effects in multicellular organisms. Human commensal E. coli are capable of synthesizing colanic acid, however colanic acid biosynthesis in E. coli peaks at around 20° C., and diminishes as temperature increases, regardless of nutrient availability. Accordingly, there is a need to develop methods for inducing colanic acid production under various conditions, including at human physiological temperatures.

Additionally, age-related metabolic dysfunction represents a substantial challenge to leading longer and healthier lives. Current therapeutic options are limited, and many address the symptoms of age-related metabolic dysfunction, such as elevated LDL cholesterol and hyperinsulinemia, rather than the underlying causes of the dysfunction. Accordingly, there is a need to develop new methods for treating age-related metabolic dysfunction.

### SUMMARY

In one aspect, the present disclosure provides a method of inducing colanic acid production in E. coli comprising contacting an E. coli bacterium with cephaloridine at a concentration of about 1.0 μg/ml to about 3.5 μg/ml, wherein the cephaloridine concentration is below the minimum inhibitory concentration for growth of the E. coli, and wherein ZraS mediates the induction of colanic acid production in the E. coli.

In another aspect, the present disclosure provides a method of inducing ZraS-mediated colanic acid production in E. coli comprising contacting an E. coli bacterium with cephaloridine at a concentration of about 1.0 μg/ml to about 3.5 μg/ml, wherein the cephaloridine concentration is below the minimum inhibitory concentration for growth of the E. coli bacterium.

In a different aspect, the present disclosure provides a method of activating PBP1a-mediated ZraS autophosphorylation in E. coli comprising contacting an E. coli bacterium with cephaloridine at a concentration of about 1.0 μg/ml to about 3.5 μg/ml, wherein the cephaloridine concentration is below the minimum inhibitory concentration for growth of the E. coli bacterium.

In any of the preceding embodiments, the cephaloridine is at a concentration of about 1.8 μg/ml. In any of the preceding embodiments, contacting the E. coli bacterium with cephaloridine increases colanic acid production by about 1.5-fold to about 6-fold compared to a E. coli bacterium not contacted with cephaloridine. In any of the preceding embodiments, the E. coli bacterium further comprises a mutation of PBP4. In any of the preceding embodiments, the E. coli bacterium is part of a microbiome within a mammalian subject and wherein contacting the E. coli bacterium with cephaloridine at a concentration of about 1.0 μg/ml to about 3.5 μg/ml comprises administering cephaloridine to the mammalian subject at about 0.225 mg/kg body weight of the mammalian subject to about 0.6 mg/kg body weight of the mammalian subject. In some embodiments, the cephaloridine is administered orally. In some embodiments, the E. coli bacterium is a human commensal E. coli bacterium. In some embodiments, the E. coli bacterium is MG1655.

In some embodiments, the method further comprises culturing the E. coli bacterium contacted with cephaloridine to generate an enhanced colanic acid E. coli culture. In some embodiments, contacting the E. coli bacterium with cephaloridine does not cause a growth delay when culturing the E. coli bacterium contacted with cephaloridine.

In some embodiments, the method further comprises purifying colanic acid from the E. coli bacterium. In some embodiments, the method further comprises a step of formulating the E. coli as a probiotic. In some embodiments, formulating the E. coli as a probiotic comprises combining the E. coli with a pharmaceutically acceptable carrier and/or a preservative.

In one aspect, the present disclosure provides a method of inducing ZraS-mediated activation of a cps operon in an E. coli bacterium that is part of a mammalian subject microbiome comprising administering cephaloridine to the mammalian subject at about 0.5 mg/kg body weight of the mammalian subject to about 1.3 mg/kg body weight of the mammalian subject. In some embodiments, the cephaloridine is administered to the mammalian subject orally.

In one aspect, the present disclosure provides a method for treating age-related metabolic dysfunction in a subject in need thereof comprising administering a therapeutically effective amount of cephaloridine. In another aspect, the present disclosure provides a method for treating age-related elevated low density lipoprotein (LDL) in a subject in need thereof comprising administering a therapeutically effective amount of cephaloridine. In a different aspect, the present disclosure provides a method for treating age-related elevated insulin in a subject in need thereof comprising administering a therapeutically effective amount of cephaloridine.

In some embodiments, the therapeutically effective amount of cephaloridine is about 0.25 mg/kg to about 0.67 mg/kg. In some embodiments, the cephaloridine is administered orally. In some embodiments, the method reduces the subject's insulin levels relative to an untreated subject. In some embodiments, the method reduces the subject's LDL levels relative to an untreated subject. In some embodiments, the method does not reduce the subject's high density lipoprotein (HDL) levels relative to an untreated subject. In some embodiments, the method reduces the subject's LDL:HDL ratio compared to an untreated subject. In some embodiments, the method reduces the subject's insulin levels and LDL:HDL ratio compared to an untreated subject.

In some embodiments, the therapeutically effective amount of cephaloridine contacts a commensal microorganism that is part of the subject's microbiota. In some embodiments, the cephaloridine contacted microorganism produces colanic acid. In some embodiments, the microorganism is E. coli.

In some embodiments, the method further comprises administering an additional therapeutic agent or intervention to the subject simultaneously, separately, or sequentially. In some embodiments, the additional therapeutic or intervention is selected from the group consisting of caloric restriction, resistance training, senolytic drug, and senomorphic drug. In some embodiments, the senolytic drug is selected from the group consisting of Navitoclax, ABT-737, BRD-K20733377, BRD-K56819078, BRD-K44839765, s63845, EF24, A1331852, A1155463, Fisetin, Quercetin, Hyperoside, 17-DMAG, Gingerenone A, 6-shogalol, UBX0101, FOXO4-DRI, P5901, P22077, Nintedanib, Proscillaridin, Ouabain, Digoxin, Oleandrin, 25-hydroxychloroquine, R406, Verteporfin, Procyanidin C1, Piperlongumine, GL-V9, TPPa derivatives, RSL3, Oridonin, Bortezomib, Azithromycin, Roxithromycin, Roxithromycin, Panobinostat, CUDC-907, Chloroquine, Bafilomycin A, JQ1, OTX015, arv825, Zoledronate, Fenofibrate, and CGP-74514A. In some embodiments, the senomorphic drug is selected from the group consisting of SB203580, UR-135756, BIRB 796, resveratrol, apigenin, wogonin, kaempferol, metformin, cortisol, corticosterone, NDGA, rapamycin, and ruxolitinib.

### BRIEF DESCRIPTION OF THE DRAWINGS

FIGS. 1A-1E show that a low dose of cephaloridine induces colanic acid biosynthesis and that colanic acid promotes longevity in C. elegans. FIG. 1A is an image of a disk diffusion assay for antibiotic treatment of SG20781, an E. coli strain that harbors a LacZ reporter for the cps operon transcriptional regulation. SG20781 treated with chemical compounds through filter paper show various blue coloration from chemical-induced cps activation. Ctrl (LB medium), Cepha (cephaloridine), CephaT (cephalothin), Cefaz (Cefazolin), Cefu (cefuroxime), PenG (penicillin G), Amp (ampicillin), Carb (carbenicillin), Col (colistin), Cipro (ciprofloxacin), Azit (azithromycin), Tet (tetracycline), and Chlor (chloramphenicol). FIG. 1B is a bar graph showing that the Alon deletion mutant in E. coli significantly increases colanic acid production by 11.5-fold compared to an untreated wildtype control, while wildtype E. coli produces 6-fold more colanic acid when treated with cephaloridine (1.8 μg/mL, Ctrl+Cepha), 3.25-fold more colanic acid when treated with penicillin G (5 μg/mL, Ctrl+PenG), and 3.5-fold more colanic acid with treated with cephalothin (0.625 μg/mL, Ctrl+CephaT). FIG. 1C is a bar graph that shows the colanic acid production from wild-type E. coli treated with a range of cephaloridine doses, with 1.8 μg/mL causing the most production of colanic acid (orange). FIG. 1D is a graph showing that low-dose cephaloridine treatment of E. coli (orange) does not alter bacterial growth (dotted lines) as compared to vehicle-treated E. coli (black) and Alon mutant E. coli (red), and that low-dose cephaloridine treatment induces colanic acid biosynthesis during the exponential and stationary growth phases (solid lines). FIG. 1E is a graph showing that the consumption of low-dose cephaloridine-treated wildtype E. coli (1.8 μg/mL, orange) promotes host C. elegans longevity by 14% compared to vehicle (black). In FIGS. 1B-1C error bars represent the mean±standard error of the mean (s.e.m.) with a minimum of three biologically independent replicates (N>3). Statistical analysis was done by two-tailed Student's t-test, **p<0.01, *p<0.05, ns means not significant. In FIG. 1D, N=2 biologically independent replicates. In FIG. 1E, N=3 biologically independent replicates, 60-100 worms per replicate, ***p<0.001 by log-rank test.

FIGS. 2A-2B show cps operon activation in SG20781, an E. coli strain that harbors a LacZ reporter for the cps operon transcriptional regulation. FIG. 2A is images of cpsB::lacZ E. coli treated by chemical compounds through filter paper (10 μg/disk or 100 μg/disk) with blue coloration from chemical-induced cps activation. FIG. 2B summarizes the treatments and cps induction, with the intensity of cps induction reflected by blue coloration and the size of the blue ring measured upon different antibiotic treatments.

FIGS. 3A-3F show that a low dose of cephaloridine promotes cps expression by commensal E. coli in a mouse gut. FIG. 3A shows that low-dose cephaloridine treated MG1655 E. coli significantly increase colanic acid production (3.2-fold increase, orange). FIG. 3B shows that the consumption of low-dose cephaloridine-treated MG1655 E. coli promotes host C. elegans longevity by 13.3% (1.8 μg/mL, orange) compared to vehicle treated (black) E. coli.

FIG. 3C is a schematic diagram of the genetic construction of cps G-R reporter. FIG. 3D shows representative bacterial images and a graph of fluorescence from low-dose cephaloridine-treated cps G-R reporter bacteria, with increased GFP-to-RFP ratios in cephaloridine-treated (orange) bacteria compared to vehicle-treated (grey) bacteria. The representative images are of cps G-R reporter E. coli treated by vehicle (left) or cephaloridine (right). Scale bars, 5 μm in the inset.

FIG. 3E is a schematic diagram of the method of administrating cps G-R reporter bacteria and low-dose cephaloridine in wildtype B6 mice. FIG. 3F shows the results of the experiment depicted in FIG. 3E, with a graph of the GFP-to-RFP ratio for bacteria isolated from the ilium of mice receiving the cps G-R reporter strain and vehicle (left), cephaloridine at 4 μg/mL (second to the left), cephaloridine at 8 μg/mL (second to the right), and tetracycline at 10 μg/mL (right), for three hours with representative bacterial images shown below. Scale bars, 5 μm in the inset. In FIG. 3C, N>3, biological independent replicates, *p<0.05 by t.test. In FIG. 3B, N=3 biologically independent replicates, 60-100 worms per replicate, ****p<0.0001 by log-rank test. In FIGS. 3D-3F statistical analysis was done by t.test and significance are denoted by asterisks: ****p<0.0001, ns.

FIGS. 4A-4D show that low-dose cephaloridine-induced colanic acid overproduction independent of the RcsC-RcsD complex. FIG. 4A is a schematic diagram of canonical RCS phosphorylation and cps activation. FIG. 4B is a bar graph showing that a low dose of cephaloridine significantly promotes colanic acid production in the wildtype E. coli (grey), ΔrcsC (purple) and ΔrcsD (pink) mutant E. coli, but not ΔrcsA (blue) and ΔrcsB (yellow) mutant E. coli. FIG. 4C is a graph showing that low-dose cephaloridine treated ΔrcsA (blue) E. coli fail to increase host C. elegans lifespan. FIG. 4D is a graph showing that low-dose cephaloridine treated ΔrcsD (pink) E. coli significantly increase host C. elegans lifespan. In FIG. 4B, error bars represent mean±s.e.m., with a minimum of three biologically independent replicates (N≥3), **P<0.01, *P<0.05, ns means non-significant, by two-tailed Student's t-test. In FIGS. 4C-4D, N=3, biological independent replicates, 60-100 worms per replicate, ****P<0.0001 by log-rank test.

FIGS. 5A-5K show that PBP1a and histidine kinase ZraS-mediate low-dose cephaloridine-induced colanic acid overproduction. FIG. 5A is a schematic diagram of PBP protein binding preference of cephaloridine and penicillin G. FIG. 5B is a bar graph showing that the loss of mrcA (PBP1a) in E. coli suppresses colanic acid induction upon low-dose cephaloridine. FIG. 5C is a workflow of transcriptional profiling in wildtype E. coli upon treatment by a low-dose cephaloridine or vehicle. FIG. 5D is a plot showing a transcriptome analysis of the results of the experiment depicted in FIG. 5C, which reveals differentially expressed genes in E. coli treated by low-dose cephaloridine (oranges). FIG. 5E is a diagram showing RcsB targeted genes enriched in RNAseq analysis. FIG. 5F is a diagram showing RstBA two-component system targeted genes enriched in the RNAseq analysis. FIG. 5G is a diagram showing BtsSR two-component system targeted genes enriched in the RNAseq analysis. FIG. 5H is a diagram showing GlrKR two-component system targeted genes enriched in the RNAseq analysis. FIG. 5I is a diagram showing ZraSR two-component system targeted genes enriched in the RNAseq analysis. FIG. 5J is a bar graph showing that the loss of ZraS in E. coli suppresses colanic acid induction upon low-dose cephaloridine. FIG. 5K is an illustration of the proposed model for cephaloridine induced, ZraS-mediated colanic acid production in E. coli. In FIGS. 5B and 5J, error bars represent mean±s.e.m., with a minimum of three biologically independent replicates (N≥3), **P<0.01, *P<0.05, ns means non-significant, by two-tailed Student's t-test. In FIG. 5C, DESeq2|log 2 fold change|≥0.5; P<0.05 by two-sided Wald test (control versus cephaloridine treated). In FIG. 5E, differentially expression genes from RNAseq with log 2 fold change>0.5 in red; log 2 fold change<−0.5 in blue; asterisk *p-value<0.05, −0.5<log 2 fold change<0.5 or undetectable genes are in grey.

FIGS. 6A-6D shows that cephaloridine attenuates age-related metabolic dysfunction. FIG. 6A shows plasma levels of low-density lipoprotein (LDL) increased by 2-fold between 12 and 18 months of age in control male mice, but remain nearly unchanged in mice treated orally with cephaloridine (2 μg/mL). FIG. 6B shows between 12 and 18 months of age in female mice, the increase in plasma insulin levels is lower in mice treated orally with cephaloridine (2 μg/mL) compared to controls. FIG. 6C shows plasma levels of high-density lipoprotein (HDL) are not affected by the cephaloridine treatment in male mice. FIG. 6D shows the age-related increase in the LDL:HDL ratio is suppressed by the cephaloridine treatment in male mice. **p<0.01, *p<0.05, ns, p>0.05 by t-test with Welch's correction.

FIG. 7 cps operon is enriched in human centenarian gut microbiota. Using two published centenarian gut microbiome datasets, the percentage of reads from the cps operon over total reads was calculated and compared between centenarian groups and elderly control groups.

### DETAILED DESCRIPTION

It is to be appreciated that certain aspects, modes, embodiments, variations and features of the present technology are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

### I. Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, “administration” of an agent, drug, bacterial strain or spore thereof, or composition of the present technology to a subject includes any route of introducing or delivering to a subject a compound to perform its intended function. Administration can be carried out by any suitable route, including orally, intranasally, parenterally (intravenously, intramuscularly, intraperitoneally, or subcutaneously), topically, or by inhalation. In some embodiments, the compositions of the present technology are formulated for enteric administration. In some embodiments, the compositions are formulated for oral, sublingual, or rectal delivery. In some embodiments, the compositions are formulated for use as a probiotic. In some embodiments, the compositions are formulated for use as a live biotherapeutic. As used herein, administration includes self-administration and administration by another.

As used herein, “age-related metabolic dysfunction” refers to diminished or impaired metabolic functions that are associated with aging. For example, age-related metabolic dysfunction can include a decline in metabolic rate, insulin resistance, hyperinsulinemia, elevated cholesterol levels, and in particular an elevated low density lipoprotein (LDL) to high density lipoprotein (HDL) ratio, or any combination thereof. Age-related metabolic dysfunction can manifest in numerous ways, including increased adiposity, decreased lean muscle mass, loss of muscle (sarcopenia), and sarcopenic obesity.

As used herein, the term “culturing” refers to the process of growing bacteria.

As used herein, “pharmaceutically acceptable carrier and/or diluent” or “pharmaceutically acceptable excipient” includes but is not limited to solvents, dispersion media, coatings, antifungal agents, isotonic and absorption delaying agents, and the like. In some embodiments, the pharmaceutically acceptable carrier comprises a polysaccharide, locust bean gum, an anionic polysaccharide, a starch, a protein, sodium ascorbate, glutathione, trehalose, sucrose, or pectin. In some embodiments, the polysaccharide comprises a plant, animal, algal, or microbial polysaccharide. In some embodiments, the polysaccharide comprises guar gum, inulin, amylose, chitosan, chondroitin sulphate, an alginate, or dextran. In some embodiments, the starch comprises rice starch. The use of such media and agents for biologically active substances is well known in the art. Further details of excipients are provided below. Supplementary active ingredients, such as antimicrobials, for example antifungal agents, can also be incorporated into the compositions.

As used herein, “pharmaceutically acceptable excipient” refers to substances and compositions that do not produce an adverse, allergic, or other untoward reaction when administered to an animal or a human. As used herein, the term includes all inert, non-toxic, liquid or solid fillers, or diluents that do not react with the therapeutic substance of the invention in an inappropriate negative manner, solvents, dispersion media, coatings, antibacterial and antifungal agents, isotonic and absorption delaying agents, preservatives and the like, for example liquid pharmaceutical carriers e.g., sterile water, saline, sugar solutions, Tris buffer, ethanol and/or certain oils.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease or condition, a positive control (a compound or composition known to exhibit the desired therapeutic effect) and a negative control (a subject or a sample that does not receive the therapy or receives a placebo) are typically employed.

As used herein, a “commensal bacterium” or “commensal bacteria” refer to a bacterium or bacteria that resides on or within a host (e.g., a human) without causing disease or harming the host. In some cases, commensal bacteria provide benefits to host organisms. Commensal bacteria make up a substantial portion of host microbiomes.

As used herein, the terms “decrease”, “reduced”, “reduction”, “decrease” or “inhibit” means a decrease by a statistically significant amount. For avoidance of doubt, “decrease”, “reduced”, “reduction”, “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, “expression control sequence” or “regulatory region” of a nucleic acid molecule means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, but are not limited to, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding site (IRES) elements for the creation of multigene, or polycistronic, messages, polyadenylation signals to provide proper polyadenylation of the transcript of a gene of interest and stop codons, and can be optionally included in an expression vector.

As used herein, the term “gene” means a segment of DNA that contains all the information for the regulated biosynthesis of an RNA product, including promoters, exons, introns, and other untranslated regions that control expression.

As used herein, the terms “increased”, “increase” or “enhance” or “activate” are all used herein to generally mean an increase by a statically significant amount. For the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, “minimum inhibitor concentration” or “MIC” refers to the lowest concentration of an antimicrobial that will inhibit the growth of a microorganism after an incubation. In some instances, growth inhibition is measured visually. In some instances, the incubation is overnight.

As used herein, “prevention”, “prevent”, or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample. As used herein, preventing a disease or condition, includes preventing or delaying the initiation of symptoms of the disease or condition or preventing a recurrence of one or more signs or symptoms of the disease or condition.

As used herein, “probiotic” refers to bacteria comprising a component of the transient or endogenous flora of a subject administered to confer a beneficial prophylactic and/or therapeutic effect on the subject. Probiotics are generally known to be safe by those skilled in the art. In some embodiments, “probiotics” include “live biotherapeutic products.”

As used herein the term “senolytic drug” refers to drugs that selectively eliminate senescent cells, which are old and damaged cells that have stopped dividing but remain in the body. Senescent cells accumulate with age and contribute to age-related diseases. Senolytic drugs include, but are not limited to, Navitoclax, ABT-737, BRD-K20733377, BRD-K56819078, BRD-K44839765, s63845, EF24, A1331852, A1155463, Fisetin, Quercetin, Hyperoside, 17-DMAG, Gingerenone A, 6-shogalol, UBX0101, FOXO4-DRI, P5901, P22077, Nintedanib, Proscillaridin, Ouabain, Digoxin, Oleandrin, 25-hydroxychloroquine, R406, Verteporfin, Procyanidin C1, Piperlongumine, GL-V9, TPPa derivatives, RSL3, Oridonin, Bortezomib, Azithromycin, Roxithromycin, Roxithromycin, Panobinostat, CUDC-907, Chloroquine, Bafilomycin A, JQ1, OTX015, arv825, Zoledronate, Fenofibrate, and CGP-74514A.

As used herein the term “senomorphic drugs” refers to drugs that modulate the effects of cellular senescence. Senomorphic drugs typically, but not always act by targeting the senescence-associated secretory phenotype (SASP) and other senescence-related pathways, potentially reducing the harmful effects of senescent cells without causing cell death. Senomorphic drugs include, but are not limited to, SB203580, UR-135756, BIRB 796, resveratrol, apigenin, wogonin, kaempferol, metformin, cortisol, corticosterone, NDGA, rapamycin, and ruxolitinib.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the term “therapeutic agent” is intended to mean a compound that, when present in an effective amount, produces a desired therapeutic effect on a subject in need thereof.

As used herein, “probiotic” refers to bacteria comprising a component of the transient or endogenous flora of a subject administered to confer a beneficial prophylactic and/or therapeutic effect on the subject.

As used herein “subject” and “patient” are used interchangeably. In some embodiments, the subject is an animal subject. In some embodiments, the animal subject is a mammal. In some embodiments, the mammalian subject is a human.

“Treating,” “treat,” “treated,” or “treatment” of a disease, condition, or disorder includes: (i) inhibiting the disease, condition, or disorder, i.e., arresting its development; (ii) relieving the disease, condition, or disorder, i.e., causing its regression; (iii) slowing progression of the disease, condition, or disorder; and/or (iv) inhibiting, relieving, or slowing progression of one or more symptoms of the disease, condition, or disorder.

It is to be appreciated that the various modes of treatment or prevention of medical diseases and conditions as described are intended to mean “substantial,” which includes total but also less than total treatment or prevention, and wherein some biologically or medically relevant result is achieved.

### II. The Microbiome, Bacterial Metabolism, and Colanic Acid

The gut microbiota present in the gastrointestinal tract plays a crucial role in human health and disease susceptibility [PMID: 22424233], influencing the host's neuronal functions [PMID: 33093662], immunity [PMID: 32433595], and life expectancy [PMID: 35468952]. The microbiota genetic contributions (termed microbiome) of over 2000 genera [PMID: 35790781] encode crucial enzymes that coordinate microbial metabolic production for unique microbial metabolites, host-derived secondary metabolites, and host-microbe shared metabolites [PMID: 34552221]. These microbial metabolites serve as essential nutrients for local intestinal epithelial cells [PMID: 37596118] or as signaling molecules that impact remote organs through blood circulation [PMID: 31825083]. The microbiota also orchestrates environmental changes, such as changes in diet and medication uses, to produce bioactive compounds based on ingested diets [PMID: 25545101] or alter drug efficacy to a more or less active-even toxic-compound [PMID: 26569070], resulting in both advantageous and disadvantageous outcomes for the host. Metagenomic and metabolomic advances have revealed nearly two thousand microbial-specific metabolites [https://mimedb.org/], but the understanding of the biological functions and regulatory pathways remains elusive.

The current knowledge of microbial metabolites has opened avenues for the development of microbiome-based therapeutics, including fecal microbiota transplantation, dietary prebiotics, enteral reconstitution of symbiotic bacteria, the introduction of engineered bacteria, and supplementation of microbiota-derived bioactive compounds [PMID: 34992261]. Here, a new approach is introduced, distinct from existing strategies, involving a bacteria-targeting chemical molecule to induce the biosynthesis of a beneficial metabolite from gut-residing microbiota.

Bacterial metabolism is intricately organized, with enzymes from a biosynthesizing pathway often co-transcribed in operons controlled by transcription factors responding to inputs from membrane receptors and kinases [PMID: 27514854]. Colanic acid, an extracellular polysaccharide, is synthesized by enzymes encoded in the capsular biosynthesis (cps) operon, and expression of the cps operon has been reported to be mediated by the RCS two-component system including RcsA, RcsB, and RcsC [PMID: 3888955]. Escherichia coli (E. coli), as well as other Enterobacteriaceae [PMID: 4311825], produce colanic acids for surviving under suboptimal environmental conditions, such as low growth temperature [PMID: 19139876], desiccation [PMID: 16349202], high osmolarity [PMID: 8576059], and exposure to β-lactams [PMID: 14553918]. Previously identified as a longevity-promoting microbial metabolite, colanic acid extends lifespan when introduced through genetically engineered E. coli in host C. elegans [PMID: 28622510] [PMID: 33325823]. Supplementation of purified colanic acids promotes longevity in both C. elegans and Drosophila [PMID: 28622510]. While E. coli is typically among the first commensal bacteria to inhabit the mammalian gut, colanic acid production becomes limited when the gut environment exceeds 30° C. [PMID: 19139876]. This restriction poses a challenge for conducting functional studies in mammals. The present disclosure identified a chemical compound that induces the cps operon in E. coli residing in the mouse gut. The longevity-promoting effect of colanic acid was confirmed by activating the cps operon in C. elegans. Furthermore, the surprising and non-canonical molecular mechanism underlying this chemical-inducing effect in E. coli was identified. This present disclosure demonstrates a novel microbiota-based practice-chemically targeting the existing commensal community to produce beneficial metabolic products, thereby enhancing host fitness.

### III. Methods of Inducing Colanic Acid Biosynthesis in E. coli

In one aspect, the present disclosure provides a method of inducing colanic acid production in E. coli comprising contacting an E. coli bacterium with cephaloridine at a concentration below the minimum inhibitory concentration for growth of the E. coli, and wherein ZraS mediates the induction of colanic acid production in the E. coli. In another aspect, the present disclosure provides a method of inducing ZraS-mediated colanic acid production in E. coli comprising contacting an E. coli bacterium with cephaloridine at a concentration below the minimum inhibitory concentration for growth of the E. coli bacterium. In a different aspect, the present disclosure provides a method of activating PBP1a-mediated ZraS autophosphorylation in E. coli comprising contacting an E. coli bacterium with cephaloridine at a concentration below the minimum inhibitory concentration for growth of the E. coli bacterium.

In some embodiments, the concentration of cephaloridine below the minimum inhibitory concentration is between about 0.9 μg/ml to about 3.6 μg/ml, about 1.0 μg/ml to about 3.5 μg/ml, about 1.1 μg/ml to about 3.4 μg/ml, about 1.2 μg/ml to about 3.3 μg/ml, about 1.3 μg/ml to about 3.2 μg/ml, about 1.4 μg/ml to about 3.1 μg/ml, about 1.5 μg/ml to about 3.0 μg/ml, about 1.6 μg/ml to about 2.5 μg/ml, about 1.7 μg/ml to about 2.0 μg/ml, or about 1.7 μg/ml to about 1.9 μg/ml. In some embodiments, the concentration of cephaloridine below the minimum inhibitory concentration is 1.8 μg/ml. In some embodiments, the E. coli bacterium is a commensal bacterium. In some embodiments, the E. coli bacterium is part of a host subject's microbiome and the induction of colanic acid biosynthesis occurs in the host organism. In some embodiments, the host organism is a mammal. In some embodiments, the host organism receives a dose of cephaloridine of about 1.0 μg/ml, 1.5 μg/ml, 2.0 μg/ml, 2.5 μg/ml, 3.0 μg/ml, about 3.5 μg/ml, about 4.0 μg/ml, about 4.5 μg/ml, or about 5.0 μg/ml.

In some embodiments, the E. coli bacterium is part of a microbiome within a mammalian subject and contacting the E. coli bacterium with cephaloridine comprises administering cephaloridine to the mammalian subject at about 0.225 mg/kg body weight of the mammalian subject to about 0.6 mg/kg body weight of the mammalian subject. In another aspect, the present disclosure provides a method of inducing ZraS-mediated activation of a cps operon in an E. coli bacterium that is part of a mammalian subject microbiome comprising administering cephaloridine to the mammalian subject at about 0.5 mg/kg body weight of the mammalian subject to about 1.3 mg/kg body weight of the mammalian subject. A range of body weight dosages (e.g., mg/kg) can be extrapolated from weight per volume doses (e.g., μg/ml) by multiplying the weight per volume dose by the volume of the dose consumed by the subject and dividing the resulting value by the weight of the subject. For example, if a subject consumes between 5-10 ml of a 2 μg/ml solution of an agent and the subject weighs between 30-40 g then a range can be extrapolated as follows: the maximum dose for the agent would be 0.67 mg/kg (10 ml×2 μg/ml÷30 g) and the minimum dose would be 0.25 mg/kg (5 ml×2 μg/ml÷40 g). Or, for example, if a subject consumes between 5-10 ml of a 1.8 μg/ml solution of an agent and the subject weighs between 30-40 g then a range can be extrapolated as follows: the maximum dose for the agent would be 0.6 mg/kg (10 ml×2 μg/ml÷30 g) and the minimum dose would be 0.225 mg/kg (5 ml×2 μg/ml÷40 g). Or, for example, if a subject consumes between 5-10 ml of a 4 μg/ml solution of an agent and the subject weighs between 30-40 g then a range can be extrapolated as follows: the maximum dose for the agent would be 0.13 mg/kg (10 ml×2 μg/ml÷30 g) and the minimum dose would be 0.5 mg/kg (5 ml×2 μg/ml÷40 g). In some embodiments, the cephaloridine is administered to the mammalian subject by any appropriate route of administration. In some embodiments, the cephaloridine is administered to the mammalian subject orally.

In some embodiments, the concentration of cephaloridine below the minimum inhibitory concentration induces an increase in colanic acid production by the E. coli bacterium by about 1-fold to about 10-fold compared to untreated E. coli. In some embodiments, the concentration of cephaloridine below the minimum inhibitory concentration induces an increase in colanic acid production by the E. coli bacterium by about 1.5-fold to about 6-fold. In some embodiments, the concentration of cephaloridine below the minimum inhibitory concentration induces an increase in colanic acid production by the E. coli bacterium by about 2-fold. In some embodiments, the concentration of cephaloridine below the minimum inhibitory concentration induces an increase in colanic acid production by the E. coli bacterium by about 2.5-fold. In some embodiments, the concentration of cephaloridine below the minimum inhibitory concentration induces an increase in colanic acid production by the E. coli bacterium by about 3-fold. In some embodiments the concentration of cephaloridine below the minimum inhibitory concentration induces an increase in colanic acid production by the E. coli bacterium by about 3.5-fold. In some embodiments, the concentration of cephaloridine below the minimum inhibitory concentration induces an increase in colanic acid production by the E. coli bacterium by about 4-fold. In some embodiments, the concentration of cephaloridine below the minimum inhibitory concentration induces an increase in colanic acid production by the E. coli bacterium by about 4.5-fold. In some embodiments, the concentration of cephaloridine below the minimum inhibitory concentration induces an increase in colanic acid production by the E. coli bacterium by about 5-fold. In some embodiments, the concentration of cephaloridine below the minimum inhibitory concentration induces an increase in colanic acid production by the E. coli bacterium by about 5.5-fold. In some embodiments the concentration of cephaloridine below the minimum inhibitory concentration induces an increase in colanic acid production by the E. coli bacterium by about 6-fold.

In some embodiments, the induction of colanic acid biosynthesis in E. coli by the concentration of cephaloridine below the minimum inhibitory concentration is mediated by the E. coli ZraS protein. In some embodiments, the concentration of cephaloridine below the minimum inhibitory concentration results in autophosphorylation of ZraS in the E. coli. In some embodiments, the autophosphorylation of ZraS activates the expression of the cps operon in the E. coli. In some embodiments, the cephaloridine binds to the PBP1a protein, and PBP1a induces ZraS autophosphorylation.

In some embodiments, the E. coli bacteria is a mammalian commensal bacterium. In some embodiments, the E. coli bacteria is a human commensal bacterium. In some embodiments, the E. coli bacteria is the MG1655 isolate. In some embodiments, the E. coli bacterium comprises a mutation of the PBP4 gene, including mutations that prevent or inhibit PBP4 protein activity. In some embodiments, the E. coli bacterium comprises a full or partial deletion of the PBP4 gene.

In some embodiments, the present disclosure provides methods of culturing the E. coli bacteria contacted with the concentration of cephaloridine below the minimum inhibitory concentration to generate an enhanced colanic acid E. coli culture. In some embodiments, contacting the E. coli bacterium with cephaloridine does not cause a growth delay when culturing the E. coli bacterium contacted with cephaloridine. In some embodiments, the enhanced colanic acid E. coli culture has greater levels of colanic acid than an E. coli culture that was not contacted with the concentration of cephaloridine below the minimum inhibitory concentration. In some embodiment, the enhanced colanic acid E. coli culture is formulated as a probiotic. In some embodiments, the method further comprises purifying colanic acid from the E. coli contacted with concentrations of cephaloridine below the minimum inhibitory concentration. Purification can be accomplished using standard methods well known in the art, such as ethanol precipitation-based methods as disclosed herein.

In any of the preceding embodiments, the method further comprises formulating the E. coli as a probiotic. In some embodiments, formulating the E. coli as a probiotic comprises formulating it for an intended route of administration, such as for oral administration. In some embodiments, formulating the E. coli as a probiotic comprises combining the E. coli with a pharmaceutically acceptable carrier and/or a preservative. Preservatives include, but are not limited to, sucrose, sodium ascorbate, glutathione, and cryoprotectants. Cryoprotectants include, but are not limited to a nucleotide, a disaccharide, a polyol, and a polysaccharide. In some embodiments, the cryoprotectant is selected from the group consisting of inosine-5′-monophosphate (IMP), guanosine-5′-monophosphate (GMP), adenosine-5′-monophosphate (AMP), uranosine-5′-monophosphate (UMP), cytidine-5′-monophosphate (CMP), adenine, guanine, uracil, cytosine, guanosine, uridine, cytidine, hypoxanthine, xanthine, orotidine, thymidine, inosine, trehalose, maltose, lactose, sucrose, sorbitol, mannitol, dextrin, inulin, sodium ascorbate, glutathione, and skim milk.

### IV. Age-Related Metabolic-Dysfunction and Methods of Treatment Thereof

Aging and metabolism are inextricably linked, and many age-related changes in body composition, including increased central adiposity and sarcopenia, have underpinnings in fundamental aging processes. These age-related changes are further exacerbated by a sedentary lifestyle and can be in part prevented by maintenance of activity with aging. Age-related changes are seen in individual metabolic tissues—adipose, muscle, and liver—as well as globally in older adults. Aging in humans has long been associated with increased levels of LDL and insulin. Elevated LDL, and in particular elevated LDL relative to HDL levels, and elevated insulin in aged humans each serve as markers for age related metabolic dysfunction. Therapeutic interventions such as caloric restriction, resistance training, and senolytic and senomorphic drugs can help maintain healthy metabolism with aging. However there remains a substantial need for treatments that can prevent or reverse age-related metabolic dysfunction, as indicated by lower levels of LDL and/or insulin.

In one aspect, the present disclosure provides a method for treating age-related metabolic dysfunction in a subject in need thereof comprising administering a therapeutically effective amount of cephaloridine. In another aspect, the present disclosure provides a method for treating age-related elevated low density lipoprotein (LDL) in a subject in need thereof comprising administering a therapeutically effective amount of cephaloridine. In a further aspect, the present disclosure provides a method for treating age-related elevated insulin in a subject in need thereof comprising administering a therapeutically effective amount of cephaloridine.

In any of the preceding embodiments, the therapeutically effective amount of cephaloridine is about 0.25 mg/kg to about 0.67 mg/kg. In some embodiments, the therapeutically effective amount of cephaloridine is about 0.25 mg/kg. In some embodiments, the therapeutically effective amount of cephaloridine is about 0.3 mg/kg. In some embodiments, the therapeutically effective amount of cephaloridine is about 0.35 mg/kg. In some embodiments, the therapeutically effective amount of cephaloridine is about 0.4 mg/kg. In some embodiments, the therapeutically effective amount of cephaloridine is about 0.45 mg/kg. In some embodiments, the therapeutically effective amount of cephaloridine is about 0.50 mg/kg. In some embodiments, the therapeutically effective amount of cephaloridine is about 0.55 mg/kg. In some embodiments, the therapeutically effective amount of cephaloridine is about 0.60 mg/kg. In some embodiments, the therapeutically effective amount of cephaloridine is about 0.65 mg/kg. In some embodiments, the therapeutically effective amount of cephaloridine is about 0.67 mg/kg. In some embodiments, the therapeutically effective amount of cephaloridine is about 0.1 ng/kg to about 14.9 mg/kg. In some embodiments, the cephaloridine is administered via any appropriate route of administration. In some embodiments, the cephaloridine is administered orally. In some embodiments, the therapeutically effective amount of cephaloridine is administered in a composition comprising a pharmaceutically acceptable carrier or excipient.

In any of the preceding embodiments, the method reduces the subject's insulin levels relative to an untreated subject. In some embodiments, the method reduces the subject's fasting insulin levels to at or below 10-12 μU/mL (milli-international units per liter). In some embodiments, the method is effective for treating hyperinsulinemia.

In any of the preceding embodiments, the method reduces the subject's LDL levels relative to an untreated subject. In any of the preceding embodiments, the method does not reduce the subject's high density lipoprotein (HDL) levels relative to an untreated subject. In any of the preceding embodiments, the method reduces the subject's LDL:HDL ratio compared to an untreated subject. In any of the preceding embodiments, the method reduces the subject's insulin levels and LDL:HDL ratio compared to an untreated subject. In some embodiments, the method is effective for reducing the LDL:HDL ratio to at or below about 3.5. In some embodiments, the method is effective for reducing the LDL:HDL ratio to at about 1.0. In some embodiments, the method is effective for reducing the LDL:HDL ratio to about 5 to about 3.5. In some embodiments, the method is effective for reducing the LDL levels to at or below 100 mg/dL.

In some embodiments, the therapeutically effective amount of cephaloridine contacts a microorganism that is part of the subject's microbiota. In some embodiments, the cephaloridine contacted microorganism produces colanic acid. In some embodiments, the microorganism is E. coli. In some embodiments, the cephaloridine activates expression of the cps operon in the microorganism.

In any of the preceding embodiments, the method further comprises administering an additional therapeutic agent or intervention to the subject. In some embodiments, the additional therapeutic or intervention is selected from the group consisting of caloric restriction, resistance training, senolytic drugs, and senomorphic drugs. In some embodiments, the method comprises further administering two or more of caloric restriction, resistance training, senolytic drugs, and senomorphic drugs.

In some embodiments, the additional therapeutic or intervention is caloric restriction. Calorie restriction (CR), the reduction of dietary intake below energy requirements while maintaining optimal nutrition, is the only known nutritional intervention with the potential to attenuate aging. Evidence from observational, preclinical, and clinical trials suggests the ability to increase life span by 1-5 years with an improvement in health span and quality of life. CR moderates intrinsic processes of aging through cellular and metabolic adaptations and reducing risk for the development of many cardiometabolic diseases.

In some embodiments, the additional therapeutic or intervention is resistance training. Resistance training, also known as strength training, is a type of exercise that involves working the muscles against a force or resistance. Resistance training is known to support maintaining muscle mass, improving mobility, and increasing the healthy years of life in aging individuals. This is particularly important to combat sarcopenia, the age-related loss of muscle mass.

In some embodiments, the additional therapeutic or intervention senolytic drugs.

Senolytic drugs are a class of medications that selectively eliminate senescent cells, which are old and damaged cells that have stopped dividing but remain in the body. These cells can accumulate with age and contribute to age-related diseases. Senolytic drugs include, but are not limited to, Navitoclax, ABT-737, BRD-K20733377, BRD-K56819078, BRD-K44839765, s63845, EF24, A1331852, A1155463, Fisetin, Quercetin, Hyperoside, 17-DMAG, Gingerenone A, 6-shogalol, UBX0101, FOXO4-DRI, P5901, P22077, Nintedanib, Proscillaridin, Ouabain, Digoxin, Oleandrin, 25-hydroxychloroquine, R406, Verteporfin, Procyanidin C1, Piperlongumine, GL-V9, TPPa derivatives, RSL3, Oridonin, Bortezomib, Azithromycin, Roxithromycin, Roxithromycin, Panobinostat, CUDC-907, Chloroquine, Bafilomycin A, JQ1, OTX015, arv825, Zoledronate, Fenofibrate, and CGP-74514A.

In some embodiments, the additional therapeutic or intervention is senomorphic drugs. Senomorphic drugs are a class of compounds that aim to modulate the effects of cellular senescence, rather than directly eliminating senescent cells like senolytics do. Senomorphic drugs typically act by targeting the senescence-associated secretory phenotype (SASP) and other senescence-related pathways, potentially reducing the harmful effects of senescent cells without causing cell death. Senomorphic drugs include, but are not limited to, SB203580, UR-135756, BIRB 796, resveratrol, apigenin, wogonin, kaempferol, metformin, cortisol, corticosterone, NDGA, rapamycin, and ruxolitinib.

### V. Formulations

The compositions of the present technology can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain solvents, diluents, and other liquid vehicles, dispersion or suspension aids, surface active agents, pH modifiers, isotonic agents, thickening or emulsifying agents, stabilizers and preservatives, solid binders, lubricants and the like, as suited to the particular dosage form desired. In certain embodiments, the compositions disclosed herein are formulated for administration to a mammal, such as a human.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push, or may be administered by continuous infusion.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

The active compounds can also be in micro-encapsulated form with one or more excipients as noted above. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

### EXAMPLES

The following examples are provided by way of illustration only and not by way of limitation. Those of skill in the art will readily recognize a variety of non-critical parameters that could be changed or modified to yield essentially the same or similar results. The examples should in no way be construed as limiting the scope of the present technology, as defined by the appended claims.

### Methods for the Examples

A. Bacteria strains

For antibiotic screening targeting cps expression, the SG20781 strain (MC4100 lon+cpsB10::lacZ Mu-immλ) was used. The bacteria were cultured overnight at 37° C. in Luria-Broth (LB) medium and evenly plated on X-gal (40 μg/mL) supplemented LB agar plates, and incubated overnight.

For colanic acid quantification, E. coli Keio mutants and the parental strain BW25113 were cultured overnight at 37° C. in M9 minimum medium [PMID: 28436966]. Bacteria were then removed by centrifugation and filtered for colanic acid measurement.

For longitudinal assays, E. coli Keio mutants were initially cultivated in M9 minimum medium at 37° C. for 16 hours. Subsequently, bacteria at stationary phase were reinoculated in fresh M9 medium with a low dose of cephaloridine at 37° C. for another 16 hours. A 100 μL aliquot of the bacteria culture was seeded onto each 6-cm NGM plate and maintained at 20° C. Fresh bacteria plates were prepared every other day for transfer to alive worms.

For high-resolution imaging, rph1 ilvG rfb-50 attL (Psyn135::mcherry <FRT>) attHKPR218 (cps G-R reporter strain), a genetically modified strain derived from MG1655 (genotype rph1 ilvG rfb-50), was generated. pPR218, integrated at the HK site, encodes Pcps::sfGFP and chloramphenicol resistance.

B. C. elegans Strain

Caenorhabditis elegans var. Bristol N2 strain was obtained from the Caenorhabditis Genome Center. The strain was maintained on standard nematode growth medium (NGM) agar plates seeded with corresponding bacteria at 20° C. Age synchronization of C. elegans was achieved by isolating eggs as previously described [PMID: 4858229].

### C. Mouse Colony

The female C57BL/6J mice in this study were handled and processed with the approval of the Institutional Animal Care and Use Committee at Baylor College of Medicine, following the current guidelines of the National Institutes of Health Model Procedure of Animal Care and Use.

### D. Disk-Diffusion Assay

E. coli strain SG20781 was used to assess antibiotic cps induction via disk diffusion as previously described [PMID: 14553918]. In brief, an overnight bacteria culture was spread onto LB agar containing 0.1 mg ml-1 5-bromo-4-chloro-3-indolyl-β-D-galactoside (X-gal) [17]. Filter papers were saturated with one of the following antibiotic solutions at 100 or 10 μg mL⁻¹ and subsequently air-dried: cephaloridine (C258600 Toronto Research Chemicals); Cephalothin (Sigma-Aldrich); Cefazolin (Sigma-Aldrich); Cefuroxime (Sigma-Aldrich); penicillin (Sigma-Aldrich); Ampicillin (Sigma-Aldrich); Carbenicillin (Sigma-Aldrich); Colistin (Sigma-Aldrich); Ciprofloxacin (Sigma-Aldrich); Azithromycin (Sigma-Aldrich); Tetracycline (Sigma-Aldrich); Chloramphenicol (Sigma-Aldrich). The soaked filter papers were placed onto the bacteria-seeding plates and incubated for 24 hours at 37° C.

### E. Colanic Acid Measurement

The desired bacterial strain was first inoculated and cultured in M9 minimum medium overnight at 37° C. to obtain the seeding culture. The OD 600 was measured, and the seeding bacteria were diluted to OD600=0.02 in M9 minimum medium for another 16 hours. After the incubation, bacterial cultures were collected in 50 mL tubes, spun down at 4,000 g at 4° C. for 30 min, filtered, and 25 mL of supernatant was collected per 50 ml conical tube. Ice-cold 100% ethanol (EtOH) was added to make a final 50% EtOH solution for precipitation, and the mixture was kept at 4° C. overnight. Once precipitation occurred, the liquid was carefully removed after centrifugation at 4,000 g at 4° C. for 50 min. The pellet was washed once with cold 80% EtOH and then air-dried in a hood. The pellet from every 50 mL culture was resuspended in 500 μL distilled H₂O. The solution was sonicated for 60 minutes at 37° C., followed by centrifugation at 4000 g at 4° C. for 10 minutes. A 200 μL sample was mixed with 30 μL hydrochloric acid (320331-500 mL, Sigma-Aldrich) and boiled for 2 hours. Subsequently, 60 μL 5M NaOH and 150 μL NaHCO₃/NaOH buffer were added to reach a pH of 6-8, calibrated by 1M HCl or NaOH solutions. The fucose quantification assay was then carried out using a K-FUCOSE kit (Megazyme) according to the manufacturer's protocol.

### F. Lifespan Assay

Lifespan assays were performed as previously described [PMID: 25554789]. In brief, age-synchronized C. elegans at the L1 stage were seeded onto designated bacteria lawns. Fresh bacteria-containing plates were made every other day for live C. elegans transfer. Once reaching adulthood as Day 0, worms were transferred to new bacteria plates. Death events were scored upon each transfer. Bagged worms and vulva protruding worms were censored throughout the analysis. Each assay contained 80-100 animals with 30-40 animals per 6 cm plate.

### G. High-Resolution Bacteria Imaging

In vitro imaging: The cps G-R reporter strain was cultured in M9 minimal medium. Bacteria culture was harvested at an OD600 of 0.6. For bacteria imaging, 1 μL of the culture was loaded onto a 1% low-melt agarose pad made with M9 medium [PMID: 23680982].

In vivo imaging: Co-housed female C57BL6 mice received cps G-R reporter E. coli at 1×10⁸ CFU per 100 mL through distilled drinking water. The bacteria-containing water was prepared and replaced daily for three days continuously. On day 3, the mice received an initial dose of cephaloridine (4 μg/ml or 8 μg/ml), tetracycline (10 μg/ml), or sterilized water administrated through oral gavage and continued receiving drug-containing water or sterilized water for 3 hours before euthanasia. Luminal content from the ileum was collected and processed for imaging bacteria from in vivo samples immediately after euthanasia as previously described [PMID: 34428120]. In brief, the ileum sections were collected and then finely minced by a pair of iris scissors in 2 mL 1×PBS. Samples were then vortexed for 1 minute and filtered with a sterile cell strainer (40 μm) to remove the tissue debris. The microbiota was washed 3 times with 1.5 mL PBS by centrifugation (15,000×g, 3 min) and then resuspended in 0.1 mL PBS for imaging.

### H. Transcriptional Analysis

Bacterial cells were grown to a mid-exponential phase starting from an OD600 of 0.03 in M9 minimal media and grown to an OD600 of 0.6. 1 mL of each culture was pelleted, and RNA was extracted using RNAsnap as previously described (PMID: 22821568) and column purified on Zymo Clean and Concentrator columns, per the manufacturer's protocol, with an off-column DNase I digestion step. Total mRNA was measured using Qubit, and the quality of RNA samples was assessed (RINe for all samples ranged from 7.9-8.2). Ribosomal depletion was performed with the Ribominus™ Transcriptome Isolation Kit per the manufacturer's protocol. The RNA libraries were prepared for Illumina sequencing using the Illumina stranded total RNA prep kit (Catalog No. 20040525) according to the manufacturer protocol. Libraries were sequenced on an Illumina NextSeq 550 platform with 2×37 cycle paired end reads. Bcl2fastq2 v2.20.0.422 was used to generate raw.fastq files, which were then filtered using FastP v0.12.4-g--poly_g_min_len 11-1 25. TPM calculations were done on filtered.fastq's using Salmon v1.10.0--seqBias--gcBias--allowDoveTails to the E. coli MG1655 genome (NC_000913.3) with an index Kmer size of 17. DEG analysis was done on count tables using Deseq2 v1.36.0, with size factors being estimated using estimateSizeFactors( ) and significance assessed with nbinomWaldTest( ). DEGs were visualized with pheatmap v1.0.12 and EnhancedVolcano v1.14.0 for heatmaps and volcano plots, respectively.

### I. Statistical Analysis

For all figure legends, asterisks indicate statistical significance as follows: NS, not significant (P>0.05), *P<0.05, **P<0.01, ***P<0.001, and ****P<0.0001. Data were obtained from at least three independent biological replicates unless specified otherwise. No statistical method was used to pre-determine the sample size. No data were excluded from the analyses. Two-tailed Student's t-test or one-way or two-way analysis of variance (ANOVA) with Holm-Sidak corrections were used as indicated in the corresponding figure legends. N indicates the number of biological replicates. n indicates the number of animals or technical replicates within each biological replicate. For survival analysis, statistical analyses were performed with SPSS software (IBM) using Kaplan-Meier survival analysis and log-rank test. For RNA-seq, a two-sided Wald test in R package DEseq2 was used. Figures and graphs were constructed using GraphPad Prism 7 (GraphPad Software) and Illustrator (CC 2019; Adobe).

### Example 1: Chemical Induction of Colanic Acid Biosynthesis In Vitro and In Vivo

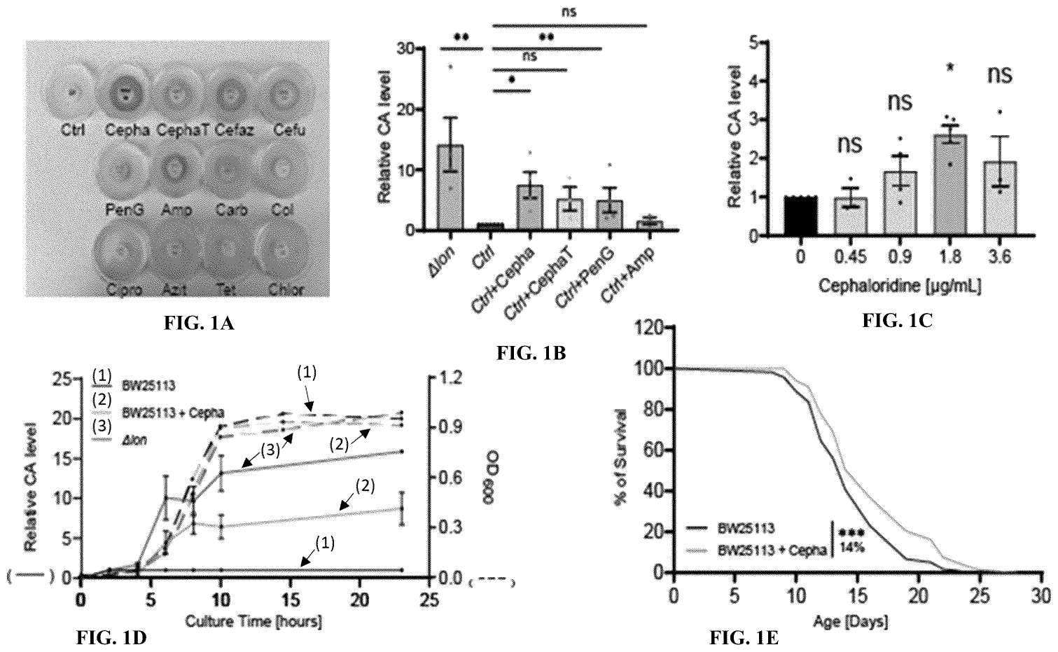

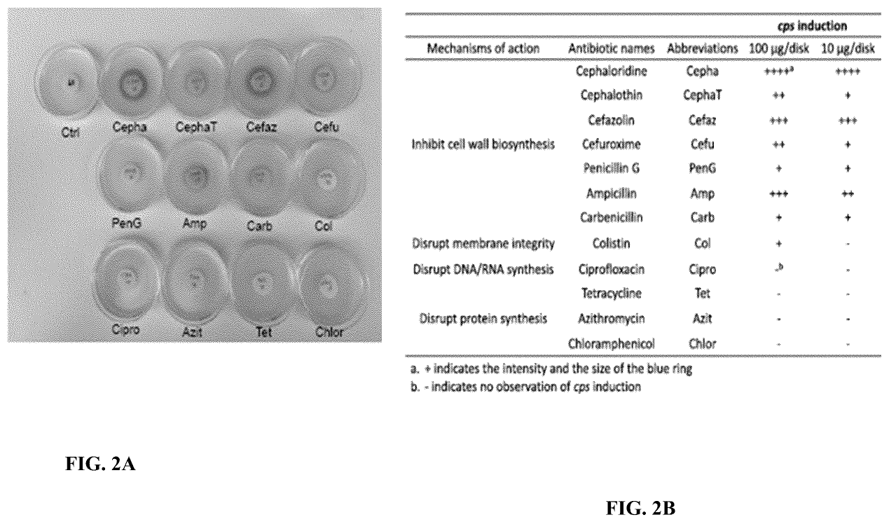

Certain β-lactam antibiotics trigger the overexpression of the cps operon in E. coli [PMID: 14553918]. To validate this finding, a disk diffusion assay was performed using SG20781, an E. coli strain that harbors a LacZ reporter for the cps operon transcriptional regulation. Using a colorimetric assay, an increased level of L-galactosidase reacts with X-gal, resulting in an intensified blue coloration as a result of the transcriptional up-regulation of the cps operon. Through screening a variety of β-lactam antibiotics as well as other types of antibiotics, it was determined that cephaloridine (Cepha), cephalothin (CephaT), cefazolin (Cafez), cefuroxime (Cefu), penicillin (PenG), ampicillin (Amp), and carbenicillin (Carb) promote various shades of blue coloration (FIGS. 1A and 2A-2B), confirming that the inhibition of peptidoglycan synthesis rather than protein synthesis or DNA replication induces the expression of the cps operon.

Next, colanic acid in the bacteria culture medium was quantified via monosaccharide-fucose-from acid-hydrolyzed polysaccharide precipitates. FIG. 1B shows that cephaloridine significantly increased colanic acid production compared to other antibiotics. FIG. 1C further shows that cephaloridine treatment at a range of below minimum inhibitory concentrations (MICs) induces colanic acid production, with 1.8 μg/mL (4.3 μM) being the optimal concentration for inducing colanic acid biosynthesis. The optimal 1.8 μg/mL value is below previously reported MIC values of 2 μg/mL (PMID 380457) and 4 μg/mL (PMID 9371340) in E. coli. Calculations were performed to determine the equivalent dosage by weight in a mammalian subject. Briefly, a typical mouse consumes 5-10 ml of water per day and weighs between 30-40 g. Using the 1.8 μg/mL concentration, a lower bound of 0.225 mg/kg (0.0018 mg/mL×5 ml=0.04 kg) and a higher bound of 0.6 mg/kg (0.0018 mg/mL×10 ml÷0.03 kg) were identified. Furthermore, FIG. 1D shows that low-dose cephaloridine did not hinder bacteria growth, which differed from the expected growth delay from using sub-MIC antibiotics [PMID: 22514370]. In particular, FIG. 1D shows that low-dose cephaloridine treated-wild-type E. coli exhibited similar growth rates to untreated E. coli and Alon E. coli mutants that overproduce colanic acid, indicating that no suppression of bacterial growth occurred from cephaloridine treatment or colanic acid production. In wild-type bacteria treated with cephaloridine and Alon E. coli mutants colanic acid production increased during the exponential growth phase and remained elevated in the stationary phase (FIG. 1D). Furthermore, FIG. 1E shows that administrating cephaloridine-treated E. coli extended the lifespan of wild-type C. elegans by 14% relative to C. elegans receiving vehicle treated control E. coli. In sum, these results highlight cephaloridine as a chemical inducer that triggers colanic acid overproduction in bacteria, ultimately promoting host longevity.

Accordingly, these results demonstrate that sub-MIC doses of cephaloridine are capable of inducing colanic acid production in E. coli, and such doses are therefore useful in methods for inducing colanic acid production in E. coli.

### Example 2: Colanic Acid Production from Commensal E. Coli in the Gut Microbiota of Mammals

Colanic acid biosynthesis in E. coli peaks around 20° C., diminishing as temperature increases, regardless of nutrient availability [PMID: 19139876]. Remarkably, the application of a low dose of cephaloridine effectively bypasses this temperature constraint, inducing colanic acid overproduction in bacteria grown at 37° C. (FIGS. 1A-1D, FIGS. 2A-2B). However, it was not clear whether this temperature independent colanic acid production would occur in commensal E. coli in the gut microbiota of mammals, where the average body temperature ranges from 36° C. to 40° C.

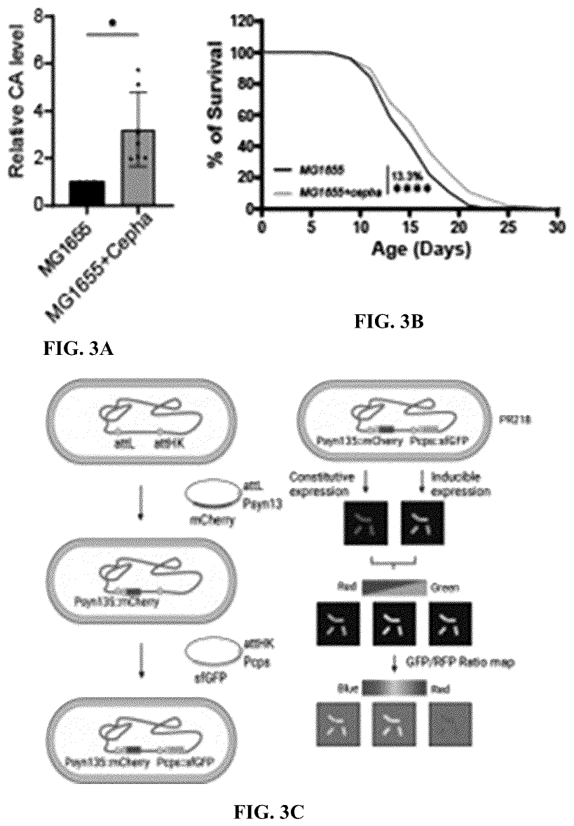

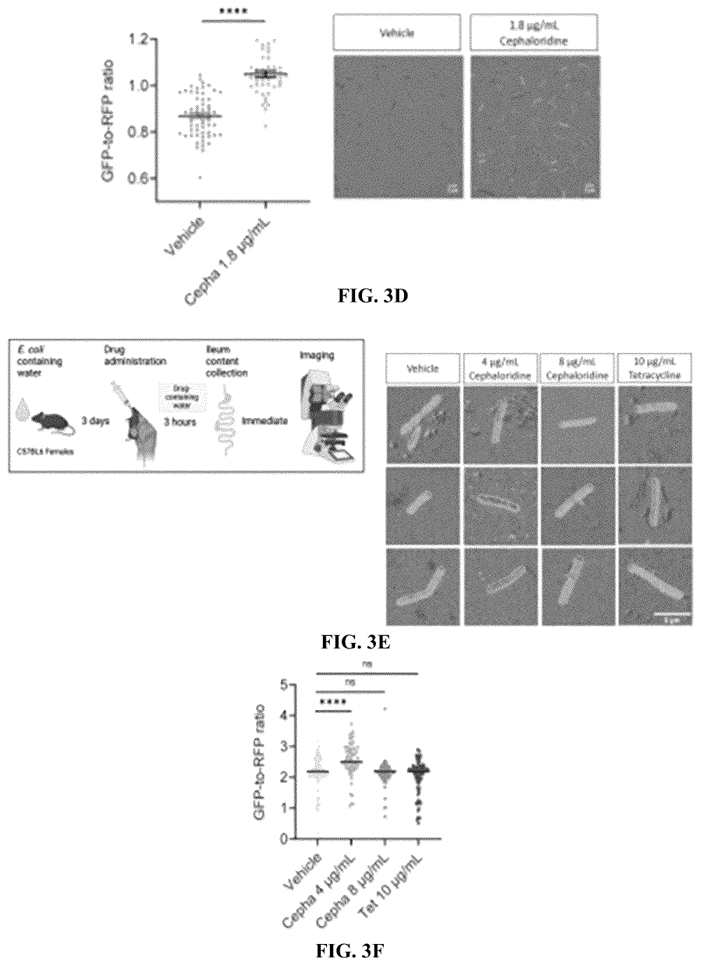

MG1655, a human-derived commensal K12 strain, was treated with a low dose of cephaloridine at 37° C., which induced colanic acid production (FIG. 3A). Furthermore, wild-type C. elegans hosting cephaloridine-treated MG1655 showed a 13% lifespan extension (FIG. 3B). To determine whether cephaloridine activates cps expression in a commensal bacterium in a mammalian host, MG1655 was engineered as shown in FIG. 3C to express GFP under the control of the cps promoter and to constitutively express RFP under a synthetic promoter syn135, resulting in the cps G-R reporter strain. FIG. 3D shows that treatment with low-dose cephaloridine caused a 20% increase in the GFP-to-RFP intensity ratio in cps G-R reporter bacteria cultured at 37° C. The cps G-R reporter strain was introduced to wild-type C57B6 mice through drinking water, followed by cephaloridine administration through a single-dose oral gavage and cephaloridine-containing water for 3 hours, which allowed for sufficient intestinal transit time [PMID: 23915679] and E. coli turnover time [PMID: 26392213] for optimal dosing (FIG. 3E). Cephaloridine, a first-generation cephalosporin classified as a β-lactam, is poorly absorbed into the host body after oral administration [PMID: 9165537]. A high dosage of cephaloridine at 8 mg/KG (1.2 mg/mL per 30 g bodyweight) has been used to treat against E. coli through intramuscular administration in systemically infected mice [PMID: 1137382]. Instead of utilizing such high doses, two lower doses of cephaloridine (4 μg/mL and 8 μg/mL) were given to induce cps operon expression in commensal E. coli, leveraging cephaloridine's minimal absorption in the intestine to achieve a localized dose. The luminal content of mice ileum was harvested after 3 hours of cephaloridine administration and were imaged for fluorescence. Imaging revealed a 13% increase in the GFP-to-RFP ratio in mice treated with 4 μg/mL cephaloridine, while, surprisingly, there was no such increase in mice treated with 8 μg/mL cephaloridine (FIG. 3F). As expected, the control group treated with tetracycline (10 μg/mL), which does not induce colanic acid production (FIGS. 1A-1B), showed no increase in the GFP-to-RFP ratio (FIG. 3F). Calculations were performed to determine the equivalent dosage by weight for mammalian subjects. Briefly, a typical mouse used in this study consumed 5-10 ml of water per day and weighed between 30-40 g. Using the 4 μg/mL concentration, a lower bound of 0.5 mg/kg (0.004 mg/mL×5 mL=0.04 kg) and a higher bound of 1.3 mg/kg (0.004 mg/mL×10 mL=0.03 kg) were identified. These results show that a low dose of cephaloridine can effectively stimulate the cps operon in E. coli within a mammalian microbiota.

Accordingly, these results demonstrate that sub-MIC doses of cephaloridine are capable of inducing E. coli cps operon activity in a mammalian commensal microbe, and such doses are therefore useful in methods for inducing colanic acid production in E. coli.

### Example 3: Molecular Mechanisms of Chemical Induction of Colanic Acid in E. Coli

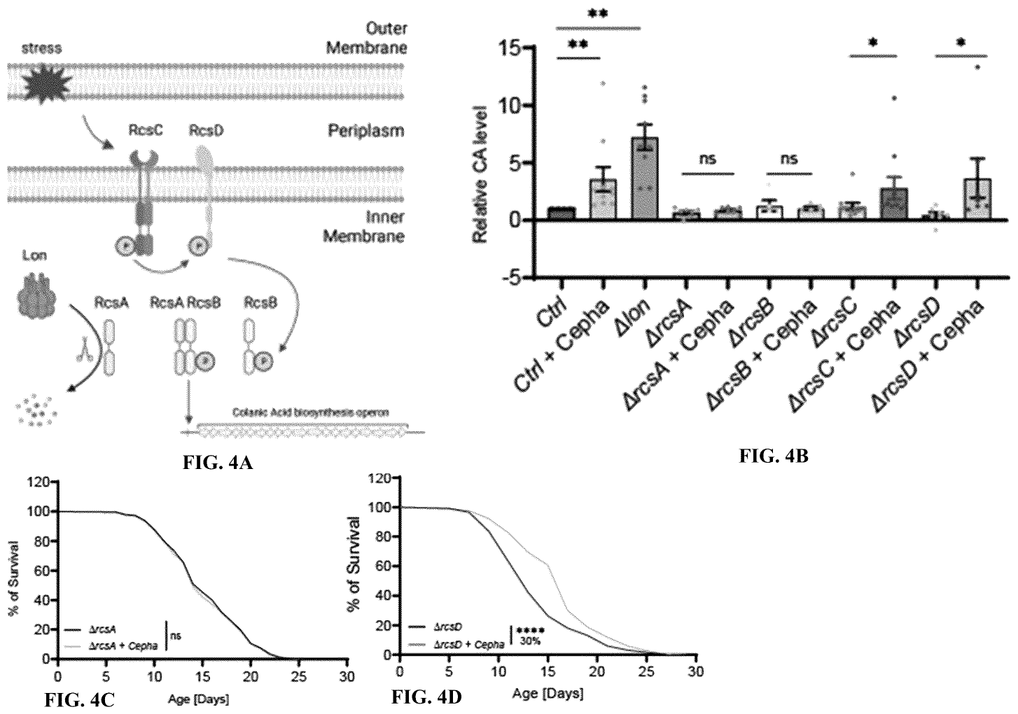

The molecular mechanism behind the induction of colanic acid by low-dose cephaloridine was investigated. It was suspected that regulation of the cps operon was centered on the well-known RCS two-component system. Canonically, the RcsC sensor histidine kinase situated in the inner membrane autophosphorylates in response to environmental cues, then the RcsD phosphotransferase transmits the phosphoryl group to the cytosolic response regulator RcsB, which functions as a transcription factor to up-regulate the cps operon, with RcsA interacting with RcsB to enhance its binding with the promoter, thereby stimulating transcriptional activation (FIG. 4A). To assess the necessity of the RCS system in colanic acid induction by low-dose cephaloridine, the E. coli deletion mutants of rcsA, rcsB, rcsC, and rcsD were treated with cephaloridine and colanic acid production was measured in their respective culture mediums. It was found that the deletion of rcsA or rcsB completely abolished the induction of colanic acid by the low dose of cephaloridine (FIG. 4B). Surprisingly, the deletion of rcsC or rcsD showed no impact on the induction of colanic acid (FIG. 4B). These results indicate that while cephaloridine relies on RcsA-RcsB to activate cps transcription, it bypasses the canonical two-component regulation mediated by RcsC-RcsD, potentially explaining its effectiveness at 37° C.

Unlike cephaloridine-treated wild-type E. coli (FIG. 1F), cephaloridine-treated rcsA deletion mutant E. coli failed to extend lifespan C. elegans (FIG. 4C). In contrast, the cephaloridine-treated rcsD deletion mutant E. coli effectively promoted longevity in C. elegans (FIG. 4D). These results reinforce that chemical induction of colanic acid in bacteria underlies the pro-longevity effect of low-dose cephaloridine on the host. Importantly, this chemical induction operates independently of the RcsC-RcsD two-component system in the bacterial inner membrane.

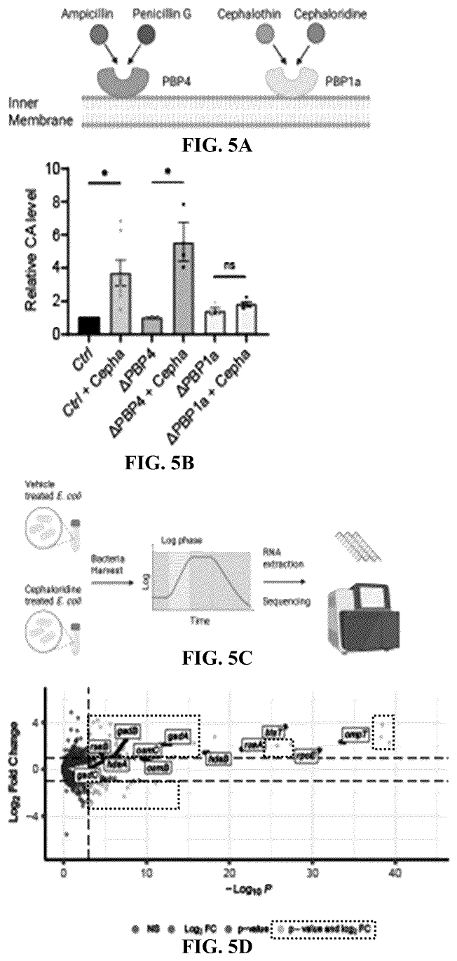

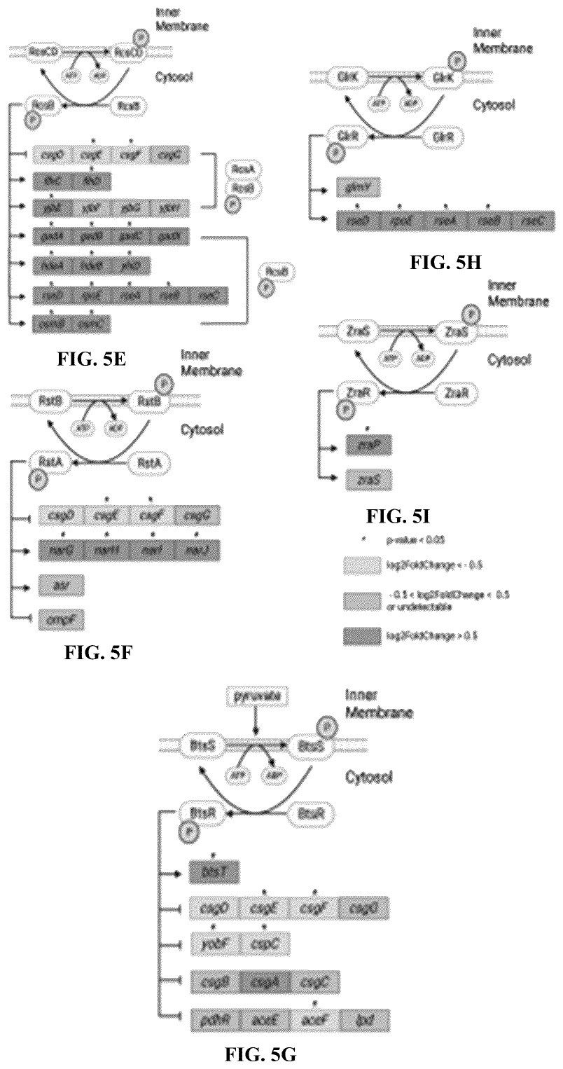

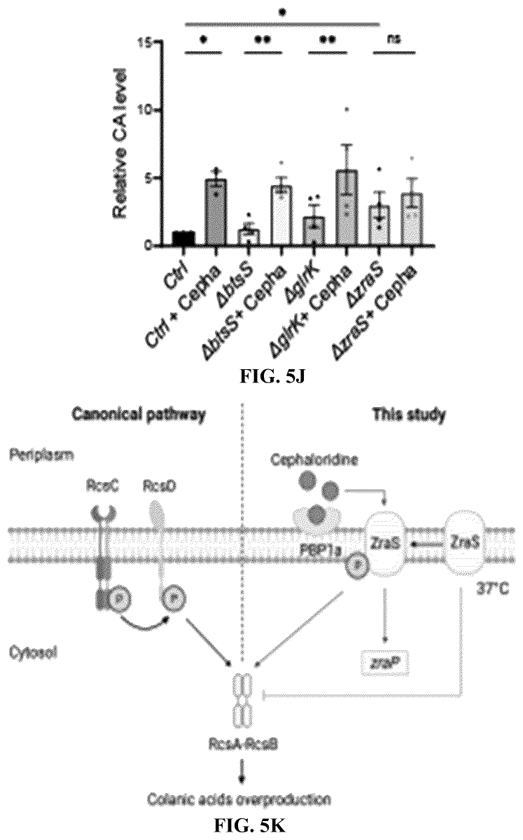

Considering β-lactam antibiotics show preferential affinities to various penicillin-binding proteins (PBPs) [PMID: 25733506] [PMID: 1103132], the role of these inner membrane proteins in colanic acid induction upon low-dose cephaloridine treatment was determined. In E. coli, a low dose of cephaloridine primarily binds to PBP1a, the protein encoded by mrcA [PMID: 7027927]. Cephalothin, another type of cephalosporin has similar binding specificity with PBP1a, whereas penicillin and ampicillin, exhibit a higher affinity for PBP4, the protein encoded by DacB [PMID: 25733506] (FIG. 5A). A low dose of cephaloridine failed to induce colanic acid in a mrcA deletion mutant (APBP1a) but did induce colanic acid production in a DacB deletion mutant (APBP4) (FIG. 5B). Surprisingly, the APBP4 mutant E. coli produced an even greater amount of colanic acid than the wild-type strain when both were treated with low dose cephaloridine (FIG. 5B). These findings show that PBP1a specifically mediates the low-dose cephaloridine chemical-induction cps effect.

A comprehensive analysis of bacterial transcriptome changes in response to the low dose of cephaloridine was conducted (FIG. 5C). Utilizing RNA-seq analysis, a set of genes were identified with significant differential expression between cephaloridine-treated and untreated E. coli (Fold change>2, P<0.05; FIG. 5D). Among these genes, an enrichment of RcsB target genes was observed (FIG. 5E), reinforcing the role of the RcsB transcription factor in the cephaloridine-mediated induction of colanic acids. Furthermore, integrating information from transcription factor and two-component system databases from RegulonDB led to the identification of four additional two-component systems—RstB-RstA (FIG. 5F), BtsS-BtsR (FIG. 5G), GlrK-GlrR (FIG. 5H), and ZraS-ZraR (FIG. 5I)—whose target genes exhibited enrichment among the differentially expressed candidate genes in response to low-dose cephaloridine.

To investigate potential mediation of colanic acid induction by these kinases, E. coli deletion mutants of btsS, glrK, and zraS were treated with low-dose cephaloridine and colanic acid levels in their respective culture mediums were measured. Due to slow growth in the M9 culture medium, the rstB deletion mutant could not be evaluated. Intriguingly, only the zras deletion mutant suppressed the induction of colanic acid by low-dose cephaloridine (FIG. 5J), suggesting ZraS as the inner membrane kinase mediating the chemical-inducing effect. Interestingly, the zraS deletion mutant showed a mild induction of colanic acid even without cephaloridine treatment compared to wild-type E. coli (FIG. 5J). Based on these results, a model was generated where dephosphorylated ZraS inhibits RcsB at 37° C., leading to the silencing of the cps operon at this temperature; but upon binding of cephaloridine to PBP1a, ZraS activation and autophosphorylation trigger the expression of the cps operon through RcsB phosphorylation (FIG. 5K).

In this disclosure a microbiota-based strategy was demonstrated that used a xenobiotic chemical to activate specific bacterial metabolic pathways, resulting in the production of a beneficial compound, colanic acid. It was determined that low-dose cephaloridine induces the cps operon and, consequently, the biosynthesis of pro-longevity colanic acid in commensal E. coli. This chemical induction overcame the natural temperature restriction on the cps expression in the mouse intestine and lead to lifespan extension in C. elegans. A 4 μg/mL dose of cephaloridine induced the cps operon in the mouse intestine, while an 8 g/mL dose failed to exert this effect. This induction effect is distinct from cephaloridine's antimicrobial property, and apart from the xenobiotic effects of the microbiota, which can alter the metabolic outcome and/or toxicity of chemical drugs that target the host [PMID: 26569070] [PMID: 28642381]. Cephaloridine exhibits extremely low oral bioavailability and its near-zero absorption in the gut has led to its discontinuation for treating infectious diseases [PMID: 9165537]. Hence, orally supplemented low-dose cephaloridine is not metabolized by bacteria and does not target host eukaryotic cells. Instead, cephaloridine acts as a chemical inducer, targeting the bacterial membrane protein PBP1a and subsequently signaling through a non-canonical ZraS-RcsA/B pathway to activate the cps operon. Inactivated ZraS may act as a phosphatase that inhibits RcsB activity through dephosphorylation. Not all antibiotics, or even other β-lactam antibiotics, exert the same effect as cephaloridine in inducing the cps operon. This indicates the significance of the chemical specificity of cephaloridine in the activation of the cps operon and the subsequent production of colanic acid.

Accordingly, these results demonstrate that sub-MIC doses of cephaloridine are capable of inducing ZraS-mediated colanic acid production in E. coli, and such doses are therefore useful in methods for inducing colanic acid production in E. coli.

### Example 4: Cephaloridine Attenuates Age-Related Metabolic Dysfunction

It is well established that LDL and insulin levels gradual increase with age in both human and mice, due to aging-related metabolic dysfunctions. Experiments were performed to determine whether cephaloridine treatment could ameliorate aging-related metabolic dysfunction as indicated by elevated LDL and insulin levels in a mouse model.

Methods: Co-housed 12-month-old C57B8 mice were given MG1655 E. coli at 1×108 CFU/mL through distilled drinking water (100 mL) for 24 hours to maintain E. coli occupancy in the mouse gut microbiota. Subsequently, mice received 2 μg/mL cephaloridine through drinking water (100 mL) for 60 hours. Matched control mice did not receive cephaloridine. This entire procedure was then repeated. Cephaloridine dosages were calculated as follows. Briefly, a typical mouse used in this study consumed 5-10 ml of water per day and weighed between 30-40 g. Using the 2 μg/mL concentration, a lower bound of 0.25 mg/kg (0.002 mg/mL×5 mL=0.04 kg) and a higher bound of 0.67 mg/kg (0.002 mg/mL×10 mL=0.03 kg) were identified.

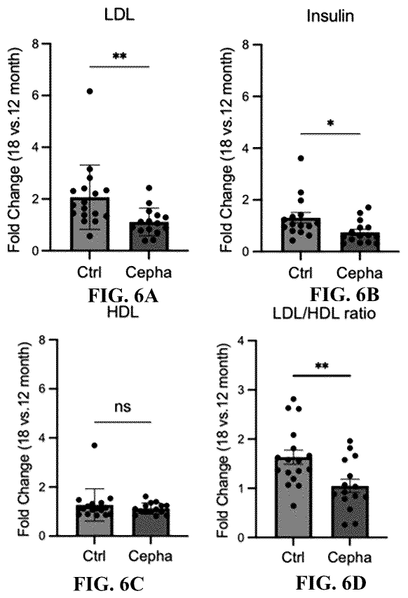

E. coli do not naturally occur in the mouse microbiota, so supplementation was necessary to allow for cephaloridine-induced colanic acid production in mice. Male mice between 12 and 18 months of age had elevated LDL levels (FIG. 6A), whereas female mice between 12 and 18 months of age had elevated insulin levels (FIG. 6B), recapitulating the age-related metabolic dysfunction phenotype. Importantly, these age-related increases were attenuated by the cephaloridine treatment during these 6 months (FIGS. 6A-6B). LDL/HDL ratio is generally considered a more accurate predictor of cardiovascular risk than absolute abundance alone. Intriguingly, cephaloridine treatment does not lower HDL level (FIG. 6C), leading to a lower LDL/HDL ratio (FIG. 6D).

Together, these new findings show that cephaloridine-induced colanic acid production mitigates age-related metabolic dysfunction in mice. Accordingly, the methods of the present disclosure are useful for treating age-related metabolic dysfunction, including age-related metabolic dysfunction associated with elevated LDL and insulin levels, age-related elevated cholesterol, and age-related elevated insulin (hyperinsulinemia) by administering cephaloridine to subjects to induce colanic acid production.

### Example 5: CPS Operon Enrichment in Older Humans

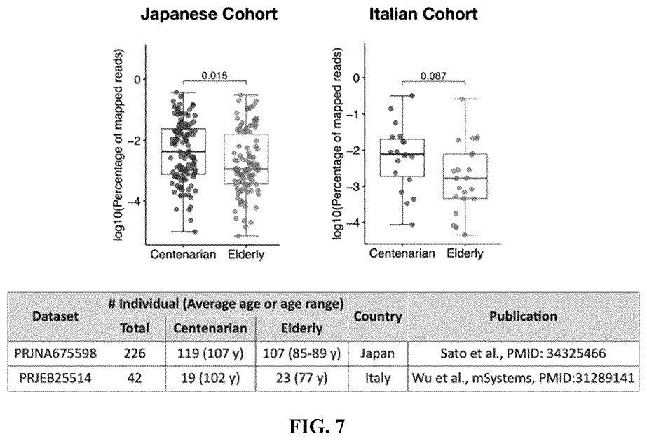

Previous studies in animal models have demonstrated the longevity-promoting effect caused by the induction of E. coli cps operon, which is responsible for the biosynthesis of colanic acid. Recently, through bioinformatic analysis, it was found that the cps operon naturally exists in the genome of human gut microbes including E. coli. To investigate whether the up-regulation of the cps operon may be associated with human longevity, two published centenarian gut microbiome datasets were analyzed. It was discovered that the cps operon is present at elevated levels in the gut microbiota of centenarians compared to elderly controls (FIG. 7). This result suggests that an approach to up-regulate the cps operon activity in elderly individuals, such as by cephaloridine administration, would recapitulate the microbiota condition observed in centenarians and, therefore, prolong human lifespan.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

Each and every publication and patent mentioned in the above specification is herein incorporated by reference in its entirety for all purposes. Various modifications and variations of the described methods and system of the present technology will be apparent to those skilled in the art without departing from the scope and spirit of the present technology. Although the present technology has been described in connection with specific embodiments, the present technology as claimed should not be unduly limited to such specific embodiments. Indeed, various modifications of the described modes for carrying out the present technology which are obvious to those skilled in the art and in fields related thereto are intended to be within the scope of the following claims.

What is claimed is:

1. A method of inducing colanic acid production in E. coli comprising contacting an E. coli bacterium with cephaloridine at a concentration of about 1.0 μg/ml to about 3.5 μg/ml, wherein the cephaloridine concentration is below the minimum inhibitory concentration for growth of the E. coli, and wherein ZraS mediates the induction of colanic acid production in the E. coli.

2. The method of claim 1, wherein, PBP1a mediates ZraS autophosphorylation in E. coli.

3. The method of claim 1, wherein the cephaloridine is at a concentration of about 1.8 μg/ml, or wherein contacting the E. coli bacterium with cephaloridine increases colanic acid production by about 1.5-fold to about 6-fold compared to a E. coli bacterium not contacted with cephaloridine, or wherein the E. coli bacterium further comprises a mutation of PBP4, or wherein the E. coli bacterium is part of a microbiome within a mammalian subject and wherein contacting the E. coli bacterium with cephaloridine at a concentration of about 1.0 μg/ml to about 3.5 μg/ml comprises administering cephaloridine to the mammalian subject at about 0.225 mg/kg body weight of the mammalian subject to about 0.6 mg/kg body weight of the mammalian subject, or wherein the E. coli bacterium is a human commensal E. coli bacterium, optionally wherein the E. coli bacterium is MG1655.

4. The method of claim 3, wherein the cephaloridine is administered to the mammalian subject orally.

5. The method of claim 1, further comprising culturing the E. coli bacterium contacted with cephaloridine to generate an enhanced colanic acid E. coli culture.

6. The method of claim 5, wherein contacting the E. coli bacterium with cephaloridine does not cause a growth delay when culturing the E. coli bacterium contacted with cephaloridine.

7. The method of claim 1, further comprising purifying colanic acid from the E. coli bacterium.

8. The method of claim 1, further comprising a step of formulating the E. coli as a probiotic.

9. The method of claim 8, wherein formulating the E. coli as a probiotic comprises combining the E. coli with a pharmaceutically acceptable carrier and/or a preservative.

10. A method of inducing ZraS-mediated activation of a cps operon in an E. coli bacterium that is part of a mammalian subject microbiome comprising administering cephaloridine to the mammalian subject at about 0.5 mg/kg body weight of the mammalian subject to about 1.3 mg/kg body weight of the mammalian subject.

11. The method of claim 10, wherein the cephaloridine is administered orally.

12. A method for treating age-related metabolic dysfunction, age-related elevated low density lipoprotein (LDL), or age-related elevated insulin in a subject in need thereof comprising administering a therapeutically effective amount of cephaloridine.

13. The method of claim 12, wherein the therapeutically effective amount of cephaloridine is about 0.25 mg/kg to about 0.67 mg/kg.

14. The method of claim 12, wherein the cephaloridine is administered orally.

15. The method of claim 12, wherein the method reduces the subject's insulin levels relative to an untreated subject, or wherein the method reduces the subject's LDL levels relative to an untreated subject, or wherein the method does not reduce the subject's high density lipoprotein (HDL) levels relative to an untreated subject, or wherein the method reduces the subject's LDL:HDL ratio compared to an untreated subject, or wherein the method reduces the subject's insulin levels and LDL:HDL ratio compared to an untreated subject.

16. The method of claim 12, wherein the therapeutically effective amount of cephaloridine contacts a commensal microorganism that is part of the subject's microbiota, optionally wherein the microorganism is E. coli.

17. The method of claim 16, wherein the cephaloridine contacted microorganism produces colanic acid.

18. The method of claim 12, further comprising administering an additional therapeutic agent or intervention to the subject simultaneously, separately, or sequentially, wherein the additional therapeutic or intervention is selected from the group consisting of caloric restriction, resistance training, senolytic drug, and senomorphic drug,
optionally wherein the senolytic drug is selected from the group consisting of Navitoclax, ABT-737, BRD-K20733377, BRD-K56819078, BRD-K44839765, s63845, EF24, A1331852, A1155463, Fisetin, Quercetin, Hyperoside, 17-DMAG, Gingerenone A, 6-shogalol, UBX0101, FOXO4-DRI, P5901, P22077, Nintedanib, Proscillaridin, Ouabain, Digoxin, Oleandrin, 25-hydroxychloroquine, R406, Verteporfin, Procyanidin C1, Piperlongumine, GL-V9, TPPa derivatives, RSL3, Oridonin, Bortezomib, Azithromycin, Roxithromycin, Roxithromycin, Panobinostat, CUDC-907, Chloroquine, Bafilomycin A, JQ1, OTX015, arv825, Zoledronate, Fenofibrate, and CGP-74514A; and
optionally wherein the senomorphic drug is selected from the group consisting of SB203580, UR-135756, BIRB 796, resveratrol, apigenin, wogonin, kaempferol, metformin, cortisol, corticosterone, NDGA, rapamycin, and ruxolitinib.
